(19) United States
(12) Patent Application Publication
(10) Pub. No.: US 2004/0258763 A1
(43) Pub. Date: Dec. 23, 2004

(54) Methods of manufacture and use of calcium phosphate particles containing allergens

(76) Inventor: Steve J.D. Bell, Marietta, GA (US)

Correspondence Address: JOHN S. PRATT, ESQ, KILPATRICK STOCKTON, LLP, 1100 PEACHTREE STREET, ATLANTA, GA, 30309, US

(21) Appl. No.: 10/824,097

(22) Filed: Apr. 13, 2004

Related U.S. Application Data
(63) Continuation-in-part of application No. 09/932,538, filed on Aug. 17, 2001.
Application No. 09/932,538 is a continuation-in-part of application No. 09/496,771, filed on Feb. 3, 2000, now Pat. No. 6,355,271.
(60) Provisional application No. 60/118,356, filed on Feb. 3, 1999.
(60) Provisional application No. 60/118,355, filed on Feb. 3, 1999.

(51) Int. Cl. A61K 39/35; A61K 39/36; A61K 9/16; A61K 9/50

(52) U.S. Cl. 424/491; 424/184.1; 424/275.1

(57) ABSTRACT

The present invention relates to the use of calcium phosphate particles in formulation with allergens for allergic desensitization. Particularly, the invention relates to novel calcium phosphate core particles, particularly nano- and micron-sized particles, as allergen adjuvants and in compositions for inducing allergic desensitization. Methods of making such particles and to methods of inducing a specific immune response using the particles of this invention are also provided.

This application is a continuation-in-part of U.S. patent application Ser. No. 09/932,538 filed on Aug. 17, 2001, which claims benefit of the filing date of U.S. patent application Ser. No. 09/496,771 filed on Feb. 3, 2000, now issued as U.S. Pat. No. 6,355,271 B and which claims benefit of the filing dates of U.S. Provisional Application Ser. Nos. 60/118,356; 60/118,364; and 60/118,355, all filed Feb. 3, 1999, the entire contents of each of which are hereby incorporated by reference.

### BACKGROUND OF INVENTION

1. Field of the Invention

The present invention relates to the use of calcium phosphate particles in formulation with allergens for allergic desensitization. Particularly, the invention relates to novel calcium phosphate core particles, particularly nano- and micron-sized particles, as allergen adjuvants and in compositions for inducing allergic desensitization. The invention also relates to methods of making such particles and to methods of inducing a preferred immune response upon encounter with allergen using the particles of this invention.

2. Description of Related Art

The World Health Organization (WHO) classifies allergy as the fourth most important disease in the world. One option for the treatment of allergies is allergic desensitization, or allergen immunotherapy—the practice of administering gradually increasing quantities of an allergen to an allergic subject to ameliorate the symptoms associated with the subsequent exposure to the allergen. Bousquet, J. et al., Annals of Allergy, Asthma, and Immunology 1998; 81:401-405.

A major problem encountered in the use of allergic desensitization, however, is the effective, yet efficient, delivery of allergens to a subject in need. Vaccine adjuvants, or agents that increases specific immune responses to an antigen, are frequently used as a vehicle for the delivery of the allergen. Goto, Norihasa; Vaccine 1994; Vol. 12, No. 6. One obstacle in the use of allergen-specific immunotherapy is finding an effective and safe adjuvant. Aluminum-containing adjuvants have historically been the preferred method of delivery because of their past superiority in allegen load. Aluminum-containing adjvants, however, occasionally produce subcutaneous (s.c.) nodules, granulomatous inflammation and sterile abscesses as local side reactions and can attract eosinophils to the injection site and enhance IgE antibody production. These reactions may persist for up to 8 weeks or sometimes longer.

The use of aluminum-containing vaccine adjuvants has other disadvantages. It has been suggested that the periodic use of vaccines adsorbed onto aluminum compounds could be related to an increased incidence of allergic diseases. Goto, Norihasa; Vaccine 1994; Vol. 12, No. 6. It is also known that aluminum adjuvanted vaccines produce a high incidence of local side reactions such as redness, pain, swelling, induration, and sterile abscess as compared with plain vaccines (i.e. vaccines containing no immunologic adjuvant). Id. Accordingly, there is a need in the art for safer and potentially more effective means of allergic densensitization.

Calcium phosphate particles have been investigated as an alternative to aluminum-containing adjuvants in parenteral vaccines and have been used in France to enhance secondary or booster immunizations against diphtheria and tetanus in humans. See Ickovic M R, Relyveld E H, Henocq E, Calcium phosphate adjuvanted allergens: Total and specific IgE levels before and after immunotherapy with house dust and mite extracts, Ann. Immunolo. (Inst. Pasteur) 1983; 134(D):385-98; Neefjes J J, Mornburg F, Cell biology of antigen presentation, Curr. Opin. Immunolo. 1993; 5(1):27-34. Calcium phosphate has also been used for allergen desensitization. See Powell M F, Newman M. J. Adjuvant properties of aluminum and calcium compounds, Vaccine Design 1995: 229-48; Relyveld, E H. Preparation and use of calcium phosphate adsorbed vaccines, Develop. Biol. Standard 1986; 65:131-136. See id.; Relyveld E H, Ickovic M R, Henocq E, Garcelon M. Calcium phosphate adjuvanted allergens. Ann Allergy 1985; 54(6):11-19. Calcium phosphate is a normal constituent of the human body and as such is well tolerated and readily resorbed. Goto, Norihasa; Vaccine 1994; Vol. 12, No. 6.

The present inventors' early studies with nanoparticulate calcium phosphate indicated that these particles produce strong Th1 T-cell-associated and mucosal IgA immunity. In strong contrast to aluminum-containing adjuvants, which generally trigger production of IgE antibody and produce local irritation at the site of injection in animal experiments and human clinical trials, CAP is cleared from the site of injection within 48 hours, does not elicit significant IgE responses, and safety and toxicity studies indicate that CAP does not trigger significant irritation at the site of injection.

Nanometer scale particles have been proposed for use as carrier particles, as supports for biologically active molecules, such as proteins, and as decoy viruses. See U.S. Pat. Nos. 5,178,882; 5,219,577; 5,306,508; 5,334,394; 5,460,830; 5,460,831; 5,462,750; and 5,464,634, the entire contents of each of which are hereby incorporated by reference. The particles disclosed in the above-referenced patents, however, are generally extremely small, in the 10-200 nm size range. Particles of this size can be difficult to make with consistency, and their morphology is not described in any detail. These patents do not disclose the use of nanoparticles as controlled release matrices Furthermore, these patents do not disclose the use of calcium phosphate particles as allergen adjuvants and as vehicles for allergens to be used for allergic desensitization.

As presented above, scientific reports have suggested a use for calcium phosphate particles as allergen adjuvants, but those calcium phosphate particles have generally been considered an unsuitable alternative to other adjuvants due to inferior adjuvanting activity. See, e.g., Goto et al., Vaccine, vol. 15, no. 12/13 (1997). One of the more important distinctions between the previously-studied calcium phosphate particles and those of the present invention is that the chemical compositions and physical characteristics of the former calcium phosphate particles is markedly different from the particles of the present invention —hence, the former's less desirable and relatively inferior adjuvanting activity. Moreover, the calcium phosphate previously evaluated was typically microparticulate (>1000 nm diameter) and possessed a rough and oblong morphology, in contrast to the smooth, spherical and colloidal core particles of the present invention.

PCT Application No. WO 00/15194 to Lee et al. published on Mar. 23, 2000 discusses calcium phosphate particles for delivery vehicles and adjuvants. This reference, however, does not provide an adequate description of the use of its particle as an allergen adjuvant. Moreover, the particles of this reference would be difficult to manufacture, because of the need for multiple steps and time-consuming, labor-intensive, and costly intervening procedures.

Calcium phosphate particles useful as core materials or carriers which can be produced simply and consistently for biologically active moieties are described in U.S. Pat. No. 6,355,271, incorporated herein by this reference. A further need remains, however, for calcium phosphate core particles that can be effectively used as adjuvants for allergens and as vehicles for the delivery of allergens to patients in need thereof in order to induce allergic desensitization.

### SUMMARY OF THE INVENTION

The present invention relates to a unique formulation of calcium phosphate (CAP) nano- and micro-particles for use as an allergen adjuvant. It further relates to a unique formulation of calcium phosphate particles in combination with allergens for use in allergic desensitization. The present inventors have found that their CAP particles provide a safer and potentially more effective means of allergic desensitization whether administered separately from the allergen or simultaneously. The inventors have found that CAP administered as a delivery vehicle and concurrently as an allergen adjuvant is a particularly suitable formulation for allergic desensitization.

More particularly, the invention relates to the core CAP particles having a diameter between about 300 nm and about 4000 nm, more particularly between about 300 nm and about 1000 nm, and having a substantially spherical shape and a substantially smooth surface, that can be combined with an allergen or allergens to a patient in need thereof in order to induce allergic desensitization.

The present invention also relates to particles having an allergen or allergens coated on the surface of the core particles, to particles having an allergen or allergens incorporated within the core particles, and to particles admixed with an allergen or allergen. The present invention further relates to methods of making these particles and to methods of using them. Non-limiting examples of suitable allergens to be at least partially coated on the surface of the core particles, incorporated within the core particles, or admixed with the core particles include one or more of the following: House Dust Mite (HDM), animal dander, molds, pollens, ragweed, latex, vespid venoms and insect-derived allergens.

The present invention also relates to combinations of this novel core particle and allergens having at least a partial coating of a surface modifying agent. If one or more of the above-mentioned allergens is at least partially coated on the particle, the agent may be optionally attached to the particle by the surface modifying agent, which acts as a biological ‘glue,’ such as cellobiose or polyethylene glycol (PEG).

One aspect of the invention generally features a method for preparing the CAP particles combined with an allergen or allergens for inducing allergic desensitization. The resulting particles may be used to elicit allergen specific immunity in a mammal by delivering an allergen or allergens in association with the CAP particles. The present invention also relates to methods of preparing the novel calcium phosphate core particles having an allergen adjuvant at least partially coated on the surface, incorporated within the core particles, or admixed with the core particles and to methods of inducing allergic desensitization by providing the particles of the present invention to a patient in need thereof. Another aspect of the invention relates to a method of treatment for allergic desensitization by delivering the particles of the present invention to a patient in need thereof.

CAP is non-toxic, non-immunogenic, and is easily degraded by the body, and accordingly, CAP can be safely administered, and administration can be repeated using the same CAP vehicle for the same or different allergens. Moreover, the CAP particles of the present invention can be prepared relatively rapidly and inexpensively.

The present inventors have developed a calcium phosphate particle that is safer and potentially more effective as vehicle and as an allergen adjuvant for the inducement of allergic desensitization. The calcium phosphate particle of the present invention has a propensity to shift Th2-biased T-cell immune responses versus allergens over towards the more desirable Th1-T-cell immune response profile versus said allergens.

The above discussed and many other features and attendant advantages of the present invention are detailed below. Other features and advantages of the invention will be apparent from the following description of the preferred embodiments thereof.

### BRIEF DESCRIPTION OF THE FIGURES

FIG. 1 is a series of schematic drawings showing various embodiments of calcium phosphate core particles. FIG. 1A shows a core particle coated directly with an allergen. FIG. 1B shows a core particle coated with surface modifying agent, such as polyethylene glycol or cellobiose, and a having an allergen adhered to the surface modifying agent. FIG. 1C shows a calcium phosphate core particle having a surface modifying agent, such as polyethylene glycol or cellobiose incorporated therein and having an allergen at least partially coating core particle.

FIG. 2 charts the degree of breathing difficulty experienced by rats injected with Allergen (HDM) combined with Alum as compared to rats injected with Allergen (HDM) combined with CaP.

### DETAILED DESCRIPTION OF SPECIFIC EMBODIMENTS OF THE INVENTION

The present invention relates to novel calcium phosphate core particles for the delivery of allergens, to methods of making them, and to methods of using the core particles as allergen delivery vehicles and allergen adjuvants inducing allergic desensitization. The present invention also relates to the novel calcium phosphate core particles having an allergen at least partially coated on the surface of the core particles, incorporated within the core particles, or admixed with the core particles, to methods of making them, and to methods of using them.

Non-limiting examples of allergens within the scope of this invention include House Dust Mite (HDM), animal dander, molds, pollens, ragweed, latex, vespid venoms and insect-derived allergens.

In addition to the CAP and an allergen, compositions of the present invention may include other components. For example, pharmaceutically acceptable buffers, preservatives, nonionic surfactants, solubilizing agents, stabilizing agents, emollients, lubricants and/or tonicity agents may be included. The compositions of the present invention may be delivered intramuscularly, parenterally, through inhalation, or across mucosal surfaces such as intraocularly, intravaginally, intranasally, and so on.

The core particles of the present invention may optionally have at least a partial coating of a surface modifying agent, which may help adhere the above-mentioned allergen or allergens to the core particle. A further aspect of the invention provides a method of treating a human or other mammal by administering a formulation as described above to a patient in need thereof.

I. Core Particles

The calcium phosphate core particles of the present invention have an average particle size between about 300 nm and about 4000 nm, more particularly, between about 300 nm and about 2000 nm. For the applications described herein, an average particle size of between about 300 nm and 1000 nm is sufficient and desirable. The core particles of the present invention have a morphology that is generally and substantially spherical in shape and a surface that is substantially smooth.

The term “substantially smooth” is used herein to mean essentially no surface features or irregularities having a size of 100 nm or larger. The core particles are colloidal in nature and may be faceted or angular and still fall within this definition, as long as the facets do not contain many surface irregularities of the type described above. The term “substantially spherical” is used herein to refer to particles that are substantially round or oval in shape, and includes particles that are relatively unfaceted and smooth, or that have very few facets, as well as particles that are polyhedral having several or numerous facets.

The following table provides a comparison between the calcium phosphate core particles of the present invention and calcium phosphate particles manufactured by Superfos Biosector a/s. The table shows that the calcium phosphate core particles of the present invention are small, smooth and ovoid, whereas Superfos Accurate CAP particles are large, jagged and crystalline.

| | BioSante | |
|---|---|---|
| | Pharmaceuticals, Inc. | Superfos Biosector a/s |
| | CAP | CAP |
| PH | 6.2-6.8 | 6.49 |
| Size | <1000 nm | >3000 nm |
| Morphology | Smooth ovoid shape | Jagged crystalline shape |

The calcium phosphate core particles of the present invention are typically prepared as a suspension in aqueous medium by reacting a soluble calcium salt with a soluble phosphate salt, and more particularly, by reacting calcium chloride with sodium phosphate under aseptic conditions. Initially, an aqueous solution of calcium chloride having a concentration between about 5 mM and about 300 mM is combined by mixing with an aqueous solution of a suitable distilled water-based solution of sodium citrate, having a concentration between about 5 mM and about 300 mM. The presence of sodium citrate contributes to the formation of an electrostatic layer around the core particle, which helps to stabilize the attractive and repulsive forces between the core particles, resulting in physically stable calcium phosphate core particles.

An aqueous solution of dibasic sodium phosphate having a concentration between about 5 mM and about 300 mM is then mixed with the calcium chloride/sodium citrate solution. Turbidity generally forms immediately, indicating the formation of calcium phosphate core particles. Mixing is generally continued for at least about 48 hours, or until a suitable core particle size has been obtained, as determined by sampling the suspension and measuring the core particle size using known methods. The core particles may be optionally stored and allowed to equilibrate for about seven days at room temperature to achieve stability in size and pH prior to further use.

In one embodiment, the calcium phosphate core particles of the present invention can be used without further modification as allergen adjuvants. In another embodiment, the core particles of the present invention can also be at least partially coated with an allergen or allergens, wherein the allergen or allergens are disposed on the surface of the core particle and optionally held in place by a surface modifying agent sufficient to bind the material to the core particle without denaturing the material.

In a further embodiment, the particles are complexed with surface modifying agents suitable for use in the present invention include substances that provide a threshold surface energy to the core particle sufficient to bind material to the surface of the core particle, without denaturing the material. Example of suitable surface modifying agents include those described in U.S. Pat. Nos. 5,460,830, 5,462,751, 5,460,831, and 5,219,577, the entire contents of each of which are incorporated herein by reference. Non-limiting examples of suitable surface modifying agents may include basic or modified sugars, such as cellobiose, or oligonucleotides, which are all described in U.S. Pat. No. 5,219,577. Suitable surface modifying agents also include carbohydrates, carbohydrate derivatives, and other macromolecules with carbohydrate-like components characterized by the abundance of —OH side groups, as described, for example, in U.S. Pat. No. 5,460,830. Polyethylene glycol (PEG) is a particularly suitable surface modifying agent.

Representative examples of two preferred CaP formulations to be used for allergic desensitization may be classed as [1] “outside” formulation; and, [2] “inside/outside” formulation.

“Outside” Formulation:

The core particles may be at least partially coated by preparing a stock solution of a surface modifying agent, such as cellobiose or PEG (e.g., around 292 mM) and adding the stock solution to a suspension of calcium phosphate core particles at a ratio of about 1 mL of stock solution to about 20 mL of particle suspension. The mixture can be swirled and allowed to stand overnight to form at least partially coated core particles. The at least partially coated core particles are administrable alone or in conjunction with one or more of the materials described below. Generally, this procedure will result in substantially complete coating of the particles, although some partially coated or uncoated particles may be present.

The various embodiments of the invention can be more clearly understood by reference to the following nonlimiting examples.

### EXAMPLE 1

A 12.5 mM solution of CaCl₂ was prepared by mixing 1.8378 g of CaCl₂ into 800 mL of sterile GDP water under aseptic conditions until completely dissolved, and the solution diluted to 1 L and filtered. A 15.625 mM solution of sodium citrate was prepared by dissolving 0.919 g of sodium citrate into 200 mL of sterile GDP water with mixing using aseptic techniques and filtered. A 12.5 mM solution of dibasic sodium phosphate was prepared by dissolving 1.775 g sodium phosphate into 1 L of sterile GDP water with mixing using aseptic techniques and filtered. All solutions were stored at room temperature.

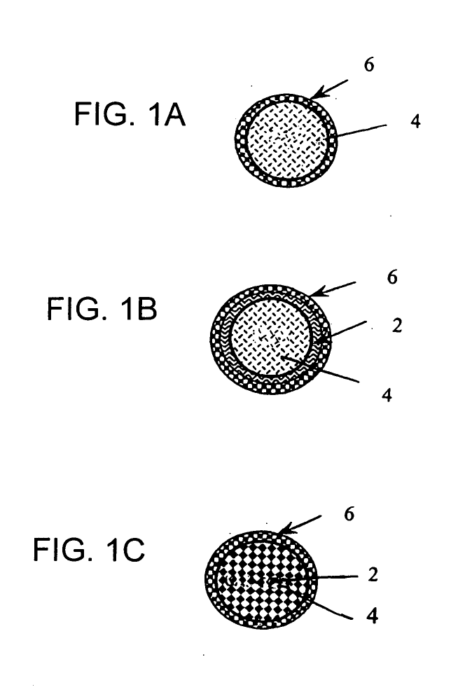

The calcium chloride solution was combined with the sodium citrate solution and thoroughly mixed. Subsequently, the sodium phosphate solution was added with mixing. Turbidity appeared immediately as particles began to form. The suspension was allowed to mix for several minutes and was sampled for endotoxin testing using aseptic technique. Mixing was continued for about 48 hours under a laminar flow hood. Following mixing, the particles were sonicated on a high power setting for about 30 minutes at room temperature, tested for endotoxin concentration and pH and characterized as to particle size with a Coulter N4Plus Submicron Particle Sizer. Photomicrographs of particles prepared in this way are shown in FIGS. 1A and 1B. Following preparation the particles were allowed to equilibrate for approximately seven days before use.

“Inside/Outside” Formulation

### EXAMPLE 2

The allergenic material was added to 75 ml or 12.5 mM calcium chloride, followed by the addition of 75 ml of 12.5 mM dibasic sodium phosphate and 15 ml of 15.6 mM sodium citrate similar to the particle formation methods described in Example 1. The solution was stirred until the final average particle size was less than 1,200 nm, as determined with a Coulter N4Plus Submicron Particle Sizer. The particle mixture containing entrapped was allergenic material was treated with cellobiose overnight and mixed again with 600 μg allergen for 1 hour at 4° C. After washing off unbounded allergen with PBS, the Allergen +CAP vaccine formulation was ready for use.

### EXAMPLE 3

The efficacy of the particles prepared as described by Example 2 was tested as follows: Three groups of 6 rats were studied. Group 1—the Control group—was immunized subcutaneously with a commercial source (ALK, Belgium) of House Dust Mite (2×10 ug-HDM) allergen without adjuvant. Group 2 was immunized subcutaneously with a commercial source (ALK, Belgium) of House Dust Mite (2×10 ug-HDM) allergen formulated with aluminum hydroxide adjuvant. Group 3 was immunized subcutaneously with a commercial source (ALK, Belgium) of House Dust Mite (2×10 ug-HDM) formulated with BioSante Pharmaceuticals calcium phosphate adjuvant. The rats received two immunizations at one-week intervals, on days 0 and 7. After completing the series of immunizations, (which usually results in the manifestation of allergic reactogenicity to the allergen), HDM allergen was instilled intratracheally (IT) in each of the three experimental groups of rats to determine the relative degrees of allergic reactivity (characterized by impaired lung function, influx of allergic cells and detection of soluble allergic mediators) after experimental challenge with HDM allergen.

### EXAMPLE 4

Allergic inflammatory responses are characterized by the occurrence of an influx of eosinophils (EOS) into the tissues where allergic reactions are occurring, and the appearance of elevated titers of allergic-specific IgE antibody in circulation. Subsequent to the experiments performed in Example 3, bronchoalveolar lavage fluid (BALF) was obtained from the lungs of the rats and H&E staining and histology was performed to quantify the relative numbers and percentages of immunological cell components isolated from the lungs.

The following tables provide the results from a study conducted to test the relative distribution (i.e. numbers) of immune cells and inflammatory mediators in Bronchoalveolar Lavage (BAL) from Rats Immunized with Allergen (HDM) combined with Alum or CaP.

| | | | | | | | |
|---|---|---|---|---|---|---|---|
| ANIMAL | #AM'S | #LYMS | #PMN'S | #EOS | PROTEIN | LDH | EPO |

A ALOH-+/−HDM “Controls”

| | | | | | | | |
|---|---|---|---|---|---|---|---|
| 1 | 155925 | 3300 | 825 | 4125 | 141 | 13 | 0.002 |
| 2 | 172575 | 11700 | 2925 | 7800 | 184 | 34 | 0.003 |
| 3 | 140800 | 12800 | 1600 | 4800 | 107 | 22 | 0.01 |
| 4 | 170925 | 7525 | 1075 | 35475 | 235 | 38 | 0.02 |
| 5 | 206400 | 3225 | 2150 | 3225 | 163 | 21 | 0.005 |
| 6 | 188600 | 8200 | 0 | 8200 | 143 | 16 | 0.002 |
| AVERAGE | 172537.5 | 7791.67 | 1429.17 | 10604.17 | 162.17 | 24 | 0.01 |
| S.DEV | 23197.06 | 4040.44 | 1031.43 | 12337.65 | 43.91 | 9.94 | 0.01 |
| S.E | 9470.16 | 1649.5 | 421.08 | 5040.91 | 17.93 | 4.06 | 0 |

B ALOH w/HDM, +HDM “Alum”

| | | | | | | | |
|---|---|---|---|---|---|---|---|
| 1 | 98600 | 8500 | 17000 | 45900 | 318 | 64 | 0.019 |
| 2 | 99750 | 9750 | 11250 | 29250 | 258 | 38 | 0.006 |
| 3 | 203300 | 11400 | 1900 | 163400 | 449 | 136 | 0.037 |
| 4 | 71775 | 11550 | 19800 | 61875 | 226 | 43 | 0.01 |
| 5 | 106575 | 8575 | 9800 | 120050 | 269 | 78 | 0.03 |
| 6 | 165750 | 10200 | 2550 | 76500 | 263 | 64 | 0.014 |
| AVERAGE | 124291.67 | 9995.83 | 10383.33 | 82829.17 | 297.17 | 70.5 | 0.02 |
| S.DEV | 49589 | 1322.54 | 7305.8 | 50181.02 | 80.06 | 35.34 | 0.01 |
| S,D, | 20244.63 | 539.92 | 2982.58 | 20486.32 | 32.68 | 14.43 | 0 |

C CAP04 w/HDM +HDM “Cap”

| | | | | | | | |
|---|---|---|---|---|---|---|---|
| 1 | 138725 | 12400 | 2325 | 1550 | 186 | 31 | 0.004 |
| 2 | 83600 | 5700 | 475 | 5225 | 223 | 28 | 0.006 |
| 3 | 148800 | 3100 | 2325 | 775 | 176 | 23 | 0.031 |
| 4 | 79650 | 2700 | 2250 | 6300 | 186 | 23 | 0.013 |
| 5 | 91854 | 6237 | 5103 | 10206 | 218 | 23 | 0.035 |
| 6 | 78806 | 2598 | 3031 | 2165 | 182 | 21 | 0.009 |
| AVERAGE | 103572.5 | 5455.83 | 2584.83 | 4370.17 | 195.17 | 24.83 | 0.02 |
| S.DEV | 31632.62 | 3747.08 | 1499.25 | 3584.78 | 20.02 | 3.82 | 0.01 |
| S,D, | 12913.96 | 1529.74 | 612.07 | 1463.48 | 8.17 | 1.56 | 0.01 |

The following tables chart the relative distribution (i.e. percentages) of immune cells and inflammatory mediators in Bronchoalveolar Lavage (BAL) from Rats Immunized with Allergen (HDM) combined with Alum or CaP.

| | | | | | | |
|---|---|---|---|---|---|---|
| ANI- | | | % | % | % | % |
| MAL | #CELLS | x20000 | AM'S | LYMS | PMN'S | EOS |

A ALOH-+/−HDM “Controls”

| | | | | | | |
|---|---|---|---|---|---|---|
| 1 | 8.25 | 165000 | 94.5 | 2 | 0.5 | 2.5 |
| 2 | 9.75 | 195000 | 88.5 | 6 | 1.5 | 4 |
| 3 | 8 | 160000 | 88 | 8 | 1 | 3 |
| 4 | 10.75 | 1215000 | 79.5 | 3.5 | 0.5 | 16.5 |
| 5 | 10.75 | 215000 | 96 | 1.5 | 1 | 1.5 |
| 6 | 10.25 | 205000 | 92 | 4 | 0 | 2 |
| AVER- | | 192500 | 89.75 | 4.17 | 0.75 | 5.25 |
| AGE | | | | | | |
| S.DEV | | 24443.81 | 5.94 | 2.46 | 0.52 | 5.59 |
| S,E | | 9979.14 | 2.42 | 1.01 | 0.21 | 228 |

B ALOH w/HDM,+HDM “Alum”

| | | | | | | |
|---|---|---|---|---|---|---|
| 1 | 8.5 | 170000 | 58 | 5 | 10 | 27 |
| 2 | 7.5 | 150000 | 66.5 | 6.5 | 7.5 | 19.5 |
| 3 | 19 | 380000 | 53.5 | 3 | 0.5 | 43 |
| 4 | 8.25 | 1650000 | 43.5 | 7 | 12 | 37.5 |
| 5 | 12.25 | 2450000 | 43.5 | 3.5 | 4 | 49 |
| 6 | 12.75 | 2550000 | 65 | 4 | 1 | 30 |
| AVER- | | 2275000 | 55 | 4.83 | 5.83 | 34.33 |
| AGE | | | | | | |
| S.DEV | | 86645.83 | 10.08 | 1.63 | 4.76 | 10.89 |
| S,D, | | 35373.01 | 4.12 | 0.67 | 1.94 | 4.45 |

C CAP04 w/HDM, +HDM “Cap”

| | | | | | | |
|---|---|---|---|---|---|---|
| 1 | 7.75 | 155000 | 89.5 | 8 | 1.5 | 1 |
| 2 | 4.75 | 95000 | 88 | 6 | 0.5 | 5.5 |
| 3 | 7.75 | 155000 | 96 | 2 | 1.5 | 0.5 |
| 4 | 4.50 | 90000 | 88.5 | 3 | 2.5 | 7 |
| 5 | 5.67 | 113400 | 81 | 5.5 | 4.5 | 9 |
| 6 | 4.33 | 86600 | 91 | 3 | 3.5 | 2.5 |
| AVER- | | 115833.33 | 89 | 4.58 | 2.33 | 4.25 |
| AGE | | | | | | |
| S.DEV | | 31717.36 | 4.87 | 2.29 | 1.47 | 3.45 |
| S,E | | 12948.56 | 1.99 | 0.93 | 0.6 | 1.41 |

These results indicate that the rats became strongly allergic after they were injected with the HDM-alum formulation. Rats receiving the HDM-alum formulation had significantly elevated numbers of allergic eosinophils as well as having significantly elevated relative percentages of allergic eosinophils relative to the rats that received the HDM-CaP formulation and showed no signs of allergic sensitization. These results strongly suggest that CaP formulated with allergens has good potential to serve as the preferred formulation for allergic desensitization (relative to the currently used aluminum adjuvant-allergen formulations).

### EXAMPLE 5

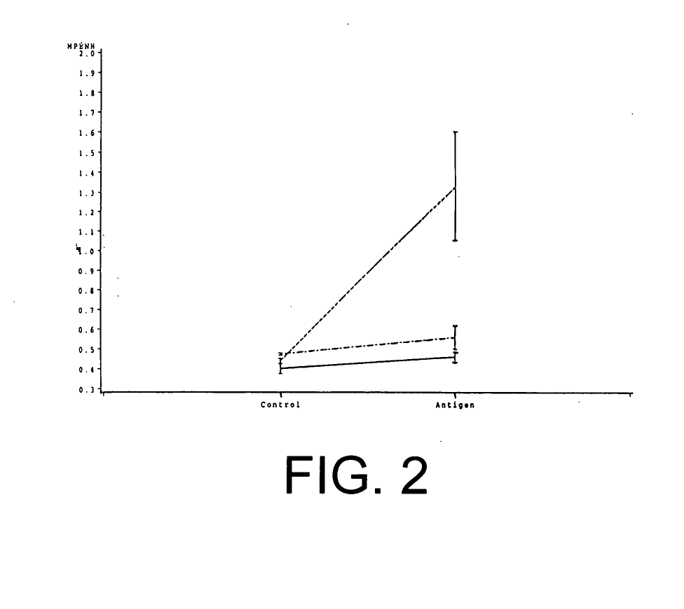

Lung function measurements were subsequently performed using whole body plethysmography (apparatus purchased from BUXCO, CT). The results are shown in FIG. 2. Data sets were derived via the integration of lung function measurements and reported in MPENH (mean enhanced pause) units on the y-axis. Higher values of MPENH indicate a greater degree of difficulty breathing. The points on the x-axis indicate the MPENH levels at the time the immunizations were given and at the time the HDM allergen was instilled intratracheally. Rats that they were injected with the HDM-alum formulation had significantly elevated MPENH values (thus, significantly greater difficulty in breathing), while the rats that were injected with the HDM-CaP formulation exhibited an elevation in MPENH values virtually identical to the rats in the control group.

The above examples generally describe methods used to evaluate three allergen formulations for impact on the lung function of rats: allergen alone, allergen with Alum, and allergen with CaP. Essentially, increased MPENH values (relative to the Control group as baseline) are indicative of impaired lung function. As indicated in FIG. 2, the rats that received the allergen with Alum formulation had significantly impaired lung function relative to the rats that received the allergen with CaP formulation. These results suggest that aluminum adjuvant in combination with allergen (i.e. the same formulation type in current usage for allergic desensitization) was seen to exacerbate allergic reactivity. In contrast, if allergen is combined with CaP (instead of alum) and administered to rats using the same allergy-inducing immunization protocol that was used to immunize with aluminum adjuvant-allergen, the CaP did not induce allergy.

What is claimed is:

1. A particle comprising calcium phosphate having an allergen at least partially coating the particle or impregnating the particle or both, wherein the particle has a diameter between about 300 nm and about 4000 nm.

2. The particle of claim 1, wherein the particle has a substantially spherical shape and a substantially smooth surface.

3. The particle of claim 1, wherein the allergen is selected from the group consisting of house dust mite, animal dander, molds, pollens, ragweed, latex, vespid venoms and insect-derived allergens., and combinations thereof.

4. The particle of claim 1, further comprising a surface modifying agent at least partially coating the particle or impregnating the particle or both.

5. The particle of claim 4, wherein the surface modifying agent comprises a basis or modified sugar.

6. The particle of claim 5, wherein the surface modifying agent comprises cellobiose.

7. The particle of claim 4, wherein the surface modifying agent comprises a carbohydrate, a carbohydrate derivative, or other macromolecule with carbohydrate-like components characterized by the abundance of —OH groups.

8. The particle of claim 4, wherein the surface modifying agent comprises polyethylene glycol.

9. The particle of claim 4, further comprising at least a partial coating of an allergen, wherein the surface modifying agent is at least partially disposed between the surface of the particle and the allergen.

10. A method for inducing an immune response in a patient, comprising delivering a particle of claim 1 to a patient in need thereof.

11. The method of claim 10, wherein the particle is delivered subcutaneously, through inhalation, or across a mucosal surface.

12. The method of claim 10, wherein the particle is complexed with a pharmaceutically acceptable excipient and delivered as a spray, an aerosol, an ointment, an eye drop, a gel, a suspension, a capsule, a suppository, an impregnated tampon, or combinations thereof.

13. A method for preparing one or more particles of claim 1, comprising reacting a soluble calcium salt with a soluble phosphate salt and an allergen.

14. The method of claim 13, wherein the soluble calcium salt comprises calcium chloride and the soluble phosphate salt comprises sodium phosphate.

15. The method of claim 14, where the reacting comprises:
(a) mixing an aqueous solution of calcium chloride with an aqueous solution of sodium citrate to form a mixture;
(b) adding an aqueous solution a sodium phosphate to the mixture to form a solution;
(c) stirring the solution until particles of the desired size and comprising calcium phosphate are obtained; and
(d) contacting the particles with an allergen to form particles that are at least partially coated with the allergen.

16. The method of claim 15, wherein the concentrations of each of the aqueous calcium chloride, the aqueous sodium citrate, and the aqueous sodium phosphate solutions are independently between about 5 mM and about 100 mM.

17. A method for preparing one or more particles of claim 4, wherein the surface modifying agent is at least partially coating the particle, comprising:
(a) adding a surface modifying agent to a suspension of calcium phosphate particles to form a mixture, and
(b) allowing the mixture to stand for sufficient time for the surface modifying agent to cover at least a portion of the particles to form at least partially coated particles.

18. The method of claim 17, wherein the surface modifying agent and suspension of calcium phosphate particles are present in a ratio of about 1:20 by volume.

19. The method of claim 18, further comprising contacting the at least partially coated particles with a solution containing an allergen to form particles that are at least partially coated with the allergen.

20. A method for preparing one or more particles comprising calcium phosphate and an allergen at least partially coating the particle and a surface modifying agent at least partially coating the particle, wherein the particle has a diameter between about 300 nm and about 4000 nm, and has a substantially spherical shape and a substantially smooth surface comprising:
(a) adding a surface modifying agent to a suspension of calcium phosphate particles to form a mixture, and
(b) allowing the mixture to stand for sufficient time for the surface modifying agent to cover at least a portion of the particles to form at least partially coated particles; and;
(c) contacting the at least partially coated particles with a solution containing an allergen to form particles that are at least partially coated with the allergen.

21. A method for treating a patient in need of allergic desensitization, comprising delivering a particle of claim 1 to a patient in need thereof.

22. The method of claim 21, wherein the particle is delivered subcutaneously, through inhalation, or across a mucosal surface.

23. The method of claim 21 wherein the particle is complexed with a pharmaceutically acceptable excipient and delivered as a spray, an aerosol, an ointment, an eye drop, a gel, a suspension, a capsule, a suppository, an impregnated tampon, or combinations thereof.

24. A method for providing a controlled release of allergen, comprising administering an effective amount of at least one particle of claim 1 to a patient in need thereof.

25. A method for inducing allergic desensitization in a patient, comprising administering an effective amount of a composition comprising:
(a) at least one particle of claim 1, and
(b) a pharmaceutically acceptable carrier solution or other excipient to a patient in need thereof.

26. A composition comprising:
(a) at least one particle of claim 1; and
(b) a pharmaceutically acceptable carrier or other excipient.
